SEQUENCE LISTING (2 sequences)

SEQ ID NO: 1 - protein, 417 aa, Homo Sapiens
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMLIPIAVGGALAGLVLIVLIAYLVGRKRSHAGYQTI

SEQ ID NO: 2 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

(19) United States
(12) Patent Application Publication
(10) Pub. No.: US 2022/0296737 A1
(43) Pub. Date: Sep. 22, 2022

(54) IMMUNOPET AND IMMUNOSPECT IMAGING TO IDENTIFY CYTOTOXIC T CELL ACTIVITY

(71) Applicant: University of Pittsburgh - Of the Commonwealth System of Higher Education, Pittsburgh, PA (US)

(72) Inventors: Carolyn Anderson, Columbia, MO (US); Wilson Edwards, Columbia, MO (US); Gary Kohanbash, Pittsburgh, PA (US)

(73) Assignee: University of Pittsburgh - Of the Commonwealth System of Higher Education, Pittsburgh, PA (US)

(21) Appl. No.: 17/686,230

(22) Filed: Mar. 3, 2022

Related U.S. Application Data
(60) Provisional application No. 63/156,590, filed on Mar. 4, 2021.

(51) Int. Cl. A61K 51/10; A61K 51/04

(52) U.S. Cl. CPC A61K 51/1093; A61K 51/1045; A61K 51/0474; A61K 2121/00; C07K 2317/515; C07K 2317/565

(57) ABSTRACT

Provided herein is a method for identifying cytotoxic T cell activity due to cancer immunotherapy in a subject. In some embodiments, the method comprises administering an effective amount of a tracer for positron emission tomography (PET) or single photon emission computed tomography (SPECT) to a subject receiving a cancer immunotherapy, wherein the tracer comprises an antibody or antigen-binding fragment thereof that specifically binds to a luminal domain of a lymphocytic granule-associated molecule (LGAM) labeled with a PET or SPECT detectable moiety, and detecting the signal of the tracer by PET or SPECT imaging to identify the cytotoxic T cell activity due to immune therapy for the cancer in the subject. In several embodiments, the antibody or antigen binding fragment specifically binds to the luminal domain of CD107a.

### CROSS REFERENCE TO RELATED APPLICATION

This application claims the benefit of priority to U.S. Provisional Application No. 63/156,590, filed Mar. 4, 2021, which is incorporated by reference herein in its entirety.

### ACKNOWLEDGMENT OF GOVERNMENT SUPPORT

This invention was made with government support under Grant No. EB029650 awarded by the National Institutes of Health. The government has certain rights in the invention.

### FIELD

This relates to embodiments of a method for identifying cytotoxic T cell activity due to cancer immunotherapy in a subject using positron emission tomography (PET) or single photon emission computed tomography (SPECT) imaging of lymphocytic granule-associated molecule (LGAM) proteins.

### BACKGROUND

Glioma is the most commonly occurring type of malignant brain tumor in adults and the leading cause of cancer-related death in children, with an average annual age-adjusted incidence rate of 6 per 100,000 population. About 30 percent of all brain and central nervous system tumors, and about 80% of all malignant brain tumors are gliomas. Clinical management is often compromised by an imprecise delineation of tumor boundaries, lack of assessment of tumor sensitivity to a given therapy, and late detection of recurrences.

Despite tumor resection and radiation therapy (with or without chemotherapy), the prognosis is dismal. Glioma patients have a 5-year survival rate of only 36%. Immunotherapy may represent a promising therapeutic strategy for gliomas; however, response rates to immunotherapies have been highly variable. As in many other cancers, glioma immunotherapy trials typically continue until disease progression is apparent, due to a lack of informative biomarkers and corresponding difficulty with early diagnosis, staging, and monitoring of the tumor.

Currently employed imaging tools, such as computed tomography (CT), magnetic resonance imaging (MRI), and PET, provide information on the localization and size of tumors (including gliomas), but are often not able to differentiate tumor homeostasis from other concurrent processes, such as inflammation, edema or bleeding. Further, assessment of tumor response to treatment using these tools is generally limited to detecting changes in tumor size and burden, delaying prognostic evaluation for weeks or months after treatment initiation. Accordingly, there is a need for non-invasive molecular imaging-based technologies that allow efficient and safe evaluation tumor response to treatment, particularly for glioma response to immunotherapy.

### SUMMARY

Provided herein is a method for identifying cytotoxic T cell activity due to cancer immunotherapy in a subject. Identifying the presence or absence of the cytotoxic T cell activity indicates whether the cancer immunotherapy is effective or not in the subject.

In several embodiments, the method comprises administering an effective amount of a tracer for PET or SPECT to a subject receiving a cancer immunotherapy. The tracer comprises an antibody or antigen-binding fragment thereof that specifically binds to a luminal domain of a LGAM and is labeled with a PET or SPECT detectable moiety. The signal of the tracer in the subject is detected by PET or SPECT to identify the cytotoxic T cell activity due to immune therapy for the cancer in the subject. In some embodiments, the method comprises quantifying and localizing the signal of the tracer in the subject.

In several embodiments, detecting an increase in the signal of the tracer in the subject compared to a control identifies the presence of cytotoxic T cell activity due to the cancer immunotherapy in the subject; and detecting no increase in the signal of the tracer in the subject compared to a control identifies the absence of cytotoxic T cell activity due to the cancer immunotherapy in the subject. In some embodiments, the method further comprising continuing the cancer immunotherapy if the presence of cytotoxic T cell activity due to immune therapy for the cancer is detected in the subject. In some embodiments, the method further comprises stopping or changing the cancer immunotherapy (for example, by providing adjuvant therapy) if the presence of cytotoxic T cell activity due to immune therapy for the cancer is not detected in the subject.

The method can be used for detection of cytotoxic T cell activity due to immunotherapy for any suitable cancer, such as for treatment of colon cancer, glioma, breast cancer, lung cancer, renal cancer, or melanoma. In several embodiments, the method is used for detection of cytotoxic T cell activity due to immunotherapy for glioma, such as glioblastoma.

The method can be used for detection of cytotoxic T cell activity due to any suitable type of cancer immunotherapy including administering to the subject an adoptive cell therapy (such as chimeric antigen receptor (CAR) T-cell therapy, a T-cell receptor (TCR) therapy, or a tumor-infiltrating lymphocyte (TIL) therapy), a tumor vaccine, or an immune checkpoint inhibitor to treat cancer in the subject.

In some embodiments, the antibody or antigen-binding fragment thereof used in the method specifically binds to CD107a, such as 1D4B antibody, G1/139/5 antibody, huMAb1 antibody, huMAb2 antibody, or huMAb3 antibody, or an antigen binding fragment thereof, or a humanized or chimeric form thereof. In some embodiments, the PET detectable moiety comprises ⁸⁹Zr, ⁶⁴Cu, ¹⁸F, ⁶⁸Ga, ¹¹C, ⁸⁶Y, or ¹²⁴I, and the SPECT detectable moiety comprises ^{99m}Tc, ¹¹¹In, ⁶⁷Ga, ¹⁷⁷Lu, or ¹³¹I.

The foregoing and other objects, features, and advantages of the invention will become more apparent from the following detailed description, which proceeds with reference to the accompanying figures.

### BRIEF DESCRIPTION OF THE DRAWINGS

FIG. 1. Schematic illustrating T-cell killing of a tumor cell by degranulation, and LGAM (CD107a) localization to the T cell membrane during degranulation.

FIGS. 2A-2C. Anti-CD107a conjugated to PET-tracer labels tumor-localized T-cells in mice treated with checkpoint immunotherapy. Mice with syngenic GL261 gliomas injected into a single hemisphere received either saline (control) or anit-PD1 and anti-CTLA4 combination therapy (ICI; 200 mg/dose, days 17 and 20 post-tumor injection (pti)). Zr-89-anti-CD107a (tracer) was injected (i.v.) on day 20 pti and mice were imaged by PET/CT on day 23 pti. (2A) Representative PET images and quantification of standard uptake values (SUV) at the tumor site. (2B) Prolonged survival following ICI treatment, and (2C) ex vivo analysis on day 23, demonstrating increased activation of T-cells in mice treated with ICI.

FIG. 3. Zr-89-anti-CD107a uptake was assessed in the mouse model of FIG. 1, localized to the hemisphere containing the tumor, and blocked with a 10× blocking dose of unlabeled anti-CD107a.

FIG. 4. Day 21 GL261 tumor-bearing mice show uptake of i.p. injected fluorescently-labeled (AF647) anti-CD107a. Lymphocyte gated cells are shown.

### SEQUENCE LISTING

The Sequence Listing is submitted as an ASCII text file in the form of the file name “Sequence.txt’ (˜8 kb), which was created on Jun. 1, 2022, and which is incorporated by reference herein.

### DETAILED DESCRIPTION

### I. Introduction

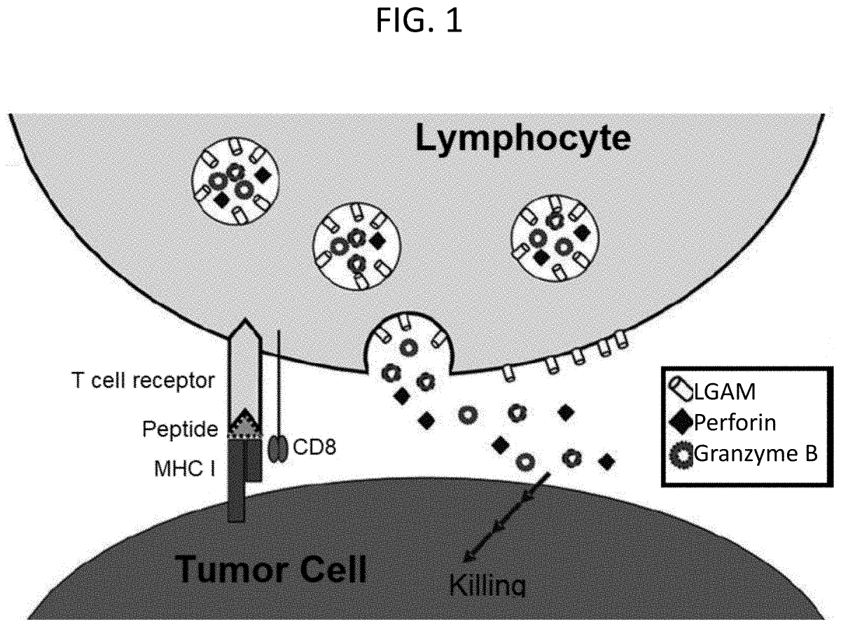

Almost all effective cancer immunotherapies converge on lymphocytes, primarily T and NK cells, releasing cytotoxic mediators at a lymphocyte-tumor synapse to mediate tumor cell killing (FIG. 1). For example, checkpoint inhibitors block inhibitory receptors and allow T-cells to elicit tumor cytotoxicity. Similarly, tumor vaccines, adoptive cell therapies, such as CAR-T and TCR, and blocking immunosuppression, such as CSF1R and IDO inhibitors, all rely on T-cells to mediated tumor killing. Tumor cell killing by T-cells occurs through T-cell degranulation, and release of cytotoxic molecules granzymes and perforin, which results in tumor cell apoptosis. During degranulation, lymphocyte granule-associate molecules (LGAMs) including CD107a, CD107b and CD63 are present on the surface of T-cells and then endocytosed back into intracellular vesicles. As described herein, LGAMs such as CD107a, are a surrogate of lymphocyte-mediated tumor killing, and are a useful marker for monitoring the efficacy of cancer immunotherapies.

Previous work in PET tracer development for monitoring immunotherapy focused on imaging immune cells generally, or T cells as marked by CD8. While these approaches may be useful for specific immunotherapies, lymphocyte degranulation is a necessary process in immunotherapy induced tumor cell death is broadly applicable across immunotherapies and cancers and provides for explicit identification of cytotoxic activity. As discussed herein, a transitory molecular target (LGAM luminal domain) associated with degranulation can be visualized by immunoPET or immunoSPECT for accurately monitoring immunotherapeutic efficacy. More specifically, it is demonstrated that the luminal domain of CD107a is a viable target for immunoPET for predicting therapeutic response to glioma immunotherapy. CD107a immunoPET informs on whether immunotherapy is eliciting tumor cell killing by T-cells, allowing for the timely altering of therapies to ultimately improve patient survivals rates.

### II. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided

About: Unless context indicated otherwise, “about” refers to plus or minus 5% of a reference value. For example, “about” 100 refers to 95 to 105.

Administration: The introduction of an agent, such as a disclosed antibody or fragment thereof, into a subject by a chosen route. Administration can be local or systemic. Exemplary routes of administration include, but are not limited to, injection (such as subcutaneous, intratumoral, intramuscular, intradermal, intraperitoneal, and intravenous), oral, sublingual, rectal, transdermal (for example, topical), intranasal, vaginal, and inhalation routes.

Antibody and Antigen Binding Fragment: An immunoglobulin, antigen-binding fragment, or derivative thereof, that specifically binds and recognizes an antigen, such as CD107a. The term “antibody” is used herein in the broadest sense and encompasses various antibody structures, including but not limited to monoclonal antibodies, polyclonal antibodies, multispecific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; minibodies; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dübel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some embodiments, a disclosed antibody includes a heterologous constant domain. For example, the antibody includes a constant domain that is different from a native constant domain, such as a constant domain including one or more modifications to increase half-life, such as by increasing binding to the neonatal Fc receptor.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from a non-human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008).

Cancer: A cancer is a biological condition in which a malignant tumor or other neoplasm has undergone characteristic anaplasia with loss of differentiation, increased rate of growth, invasion of surrounding tissue, and which is capable of metastasis. A neoplasm is a new and abnormal growth, particularly a new growth of tissue or cells in which the growth is uncontrolled and progressive. A tumor is an example of a neoplasm. Non-limiting examples of types of cancer include lung cancer, stomach cancer, colon cancer, breast cancer, uterine cancer, bladder cancer, head and neck cancer, kidney cancer, liver cancer, ovarian cancer, pancreatic cancer, prostate cancer, rectum cancer, and brain cancers such as glioma.

Cancer Immunotherapy: A treatment that stimulates the immune system of a subject to target and kill cancer cells within the subject. Active cancer immunotherapies specifically target tumor cells via the immune system. Non-limiting examples include cancer vaccines, CAR-T cells, and targeted antibody therapies. Passive cancer immunotherapies do not directly target tumor cells, but enhance the ability of the immune system to attack cancer cells. Non-limiting examples include checkpoint inhibitors and cytokines. Cancer immunotherapies activate cytotoxic T-cells within the subject to target and kill cancer cells within the subject.

CD107a and CD107b: Also known as LAMP1 and LAMP2, respectively; see the lysosomal associated membrane protein definition below.

Conjugate: A complex of two molecules linked together, for example, linked together by a covalent bond. In one embodiment, an antibody or an antigen binding fragment thereof is linked to an effector molecule or a detectable moiety (such as a radiolabeled chelator or PET tracer); for example, an antibody that specifically binds to CD107a linked to 89Zr-DFO (deferrioximine). The linkage can be by chemical or recombinant means. In one embodiment, the linkage is chemical, wherein a reaction between the antibody or antigen binding fragment thereof and the detectable moiety has produced a covalent bond formed between the two molecules to form one molecule. A peptide linker (short peptide sequence) can optionally be included between the antibody or antigen binding fragment thereof and the detectable moiety. Because conjugates can be prepared from two molecules with separate functionalities, such as an antibody and an effector molecule or an antibody and a detector moiety, they are also sometimes referred to as “chimeric molecules.”

Control: A reference standard. In some embodiments, the control is a negative control, such as sample obtained from a healthy subject, such as a patient who does not have a disease, such as a cancer, and/or who is not being treated with an immune therapy. In other embodiments, the control is a positive control, such as a tissue sample from a patient who is being treated with an immunotherapy. In still other embodiments, the control is a historical control or standard reference value or range of values (such as a previously tested control sample, such as a group of patients with known prognosis or outcome, or group of samples that represent baseline or normal values).

A difference between a test sample or subject and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Detecting: Identification of the existence, presence, or fact of something. General methods of detecting are known to the skilled artisan and may be supplemented with the protocols and reagents disclosed herein. For example, included herein are methods of detecting a cytotoxic T cell marker, CD107a. Non-limiting examples of detection methods include radiolocalization, radioimaging, positron emission tomography (e.g., using an ¹⁸F-labled antibody), magnetic resonance imaging.

Effective amount: The amount of an agent (such as an antibody or antigen binding fragment thereof linked to a detection moiety, such as a PET tracer) that alone, or together with one or more additional agents, is sufficient to achieve a desired result in vitro or in vivo. For instance, this can be the amount necessary to identify a molecule in the subject (such as CD107a), or identify cytotoxic T cell activity, such as compared to a control, by detecting a detectable moiety linked to an antibody or antigen binding fragment thereof that was administered to the subject. An effective amount can be the amount of a detectable moiety linked to an antibody or antigen binding fragment thereof necessary to identify cytotoxic T cell activity due to immune therapy, such as immune therapy to treat a cancer, in a subject.

Several preparations disclosed herein can be administered to a subject in an effective amount. When administered to a subject, a dosage will generally be used that achieves target tissue concentrations that has been shown to achieve a desired effect in vitro or in a test subject Ideally, an effective amount provides a diagnostic effect with optimal sensitivity and specificity, without causing a substantial cytotoxic effect in the subject. The effective amount administered to a subject will vary depending upon a number of factors associated with that subject, for example the overall health of the subject, and the manner of administration of the agent. An effective amount can be determined by varying the dosage and measuring the resulting response. Effective amounts also can be determined through various in vitro, in vivo or in situ assays. The disclosed agent can be administered in a single dose, or in several doses, as needed to obtain the desired response.

ImmunoPET and ImmunoSPECT: A type of PET or SPECT imaging involving administration of a tracer including an antibody or antigen binding fragment labeled with a PET or SPECT detectably moiety (e.g., a radionuclide) to the imaging subject, followed by detection and localization of the tracer by PET or SPECT imaging. ImmunoPET and immunoSPECT combine the high sensitivity and quantitative capabilities of PET and SPECT with the specificity and selectivity of antibody-based molecules for a given cell surface marker.

ImmunoPET Tracer: An antibody or antigen binding fragment linked to a detectable moiety for PET imaging (such as ⁸⁹Zr, ⁶⁴Cu, ¹⁸F, ⁶⁸Ga, ¹¹C, ⁸⁶Y, ¹²⁴I) typically by a linker containing a chelator group that chelates the radionuclide. A PET detectable moiety is any molecule suitable for detection by PET imaging of a subject.

ImmunoSPECT Tracer: An antibody or antigen binding fragment linked to a detectable moiety for PET imaging (such as ^{99m}Tc, ¹¹¹In, ⁶⁷Ga, ¹⁷⁷ Lu, ¹³¹I), typically by a linker containing a chelator group that chelates the radionuclide. A SPECT detectable moiety is any molecule suitable for detection by PET imaging of a subject.

Inhibiting or treating a disease: Inhibiting the full development of a disease or condition, for example, in a subject who is at risk for a disease such as a cancer, such as glioma. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of cancer. The beneficial effect can be evidenced, for example, by a delayed onset of clinical symptoms of the disease in a susceptible subject, a reduction in severity of some or all clinical symptoms of the disease, a slower progression of the disease, a reduction in a tumor or tumors, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology or of recurrence of the disease.

Luminal domain: The portion of a membrane protein that extends into the endosomal lumen. For membrane proteins that cycle within the endosomal system and the cell surface, the luminal domain extends into the extracellular space when the membrane protein is present on the cell surface.

Lysosomal associated membrane protein or LAMP: A family of membrane glycoproteins typically concentrated on lysosomes. Non-limiting examples include CD107a (also called LAMP-1), CD107b (also called LAMP-2), and LAMPS. An exemplary human CD107a protein sequence, including the signal peptide (positions 1-28), luminal domain (positions 29-382), and transmembrane domain and cytosolic tail (positions 383-417) is available as GenBank NP_005552.3. An exemplary human CD107b protein sequence, including the signal peptide (positions 1-28), luminal domain (positions 29-375), and transmembrane domain and cytosolic tail (positions 376-410) is available as GenBank NP_002285.1.

CD107a (GenBank NP_005552.3)
(SEQ ID NO: 1)
MAAPGSARRPLLLLLLLLLLGLMHCASAAMFMVKN
GNGTACIMANFSAAFSVNYDTKSGPKNMTFDLPSD
ATVVLNRSSCGKENTSDPSLVIAFGRGHTLTLNFT
RNATRYSVQLMSFVYNLSDTHLFPNASSKEIKTVE
SITDIRADIDKKYRCVSGTQVHMNNVTVTLHDATI
QAYLSNSSFSRGETRCEQDRPSPTTAPPAPPSPSP
SPVPKSPSVDKYNVSGTNGTCLLASMGLQLNLTYE
RKDNTTVTRLLNINPNKTSASGSCGAHLVTLELHS
EGTTVLLFQFGMNASSSRFFLQGIQLNTILPDARD
PAFKAANGSLRALQATVGNSYKCNAEEHVRVTKAF
SVNIFKVWVQAFKVEGGQFGSVEECLLDENSMLIP
IAVGGALAGLVLIVLIAYLVGRKRSHAGYQTI
CD107b (GenBank NP_002285.1)
(SEQ ID NO: 2)
MVCFRLFPVPGSGLVLVCLVLGAVRSYALELNLTD
SENATCLYAKWQMNFTVRYETTNKTYKTVTISDHG
TVTYNGSICGDDQNGPKIAVQFGPGFSWIANFTKA
ASTYSIDSVSFSYNTGDNTTFPDAEDKGILTVDEL
LAIRIPLNDLFRCNSLSTLEKNDVVQHYWDVLVQA
FVQNGTVSTNEFLCDKDKTSTVAPTIHTTVPSPTT
TPTPKEKPEAGTYSVNNGNDTCLLATMGLQLNITQ
DKVASVININPNTTHSTGSCRSHTALLRLNSSTIK
YLDFVFAVKNENRFYLKEVNISMYLVNGSVFSIAN
NNLSYWDAPLGSSYMCNKEQTVSVSGAFQINTFDL
RVQPFNVTQGKYSTAQDCSADDDNFLVPIAVGAAL
AGVLILVLLAYFIGLKHHHAGYEQF

Linker: A molecule that can be used to link two molecules together, for example, to link an antibody or antigen binding fragment to a chelator or other moiety that binds a radionuclide for PET or SPECT imaging.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired response. It may also be accompanied by medications for treatment purposes. The unit dosage form may be, for example, in a sealed vial that contains sterile contents or a syringe for injection into a subject, or lyophilized for subsequent solubilization and administration or in a solid or controlled release dosage.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a cell, for example CD107a on a cytotoxic T cell) and does not bind in a significant amount to other proteins present in the sample or subject. Specific binding can be determined by standard methods. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

With reference to an antibody-antigen complex, specific binding of the antigen and antibody typically has a K_{D} of less than about 10⁻⁷ Molar, such as less than about 10⁻⁸ Molar, 10⁻⁹, or even less than about 10⁻¹⁰ Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

It is, of course, recognized that a certain degree of non-specific interaction may occur between an antibody or antigen binding fragment thereof and a non-target. Typically, specific binding results in a much stronger association between the antibody and a target protein than between the antibody and other different proteins. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein.

Subject: Any mammal, such as humans, non-human primates, pigs, sheep, cows, rodents, and the like. In two non-limiting examples, a subject is a human subject or a murine subject. Thus, the term “subject” includes both human and veterinary subjects. In an additional example, a subject is selected that is in need of cancer immunotherapy. For example, the subject either has cancer, or is in remission from a cancer, or is at risk of a cancer and in need of treatment.

Under conditions sufficient for: A phrase that is used to describe any environment, such as an in vitro or in vivo environment, that permits a desired activity.

### III. Methods of Identifying Cytotoxic T Cell Activity Due to Cancer Immunotherapy

Embodiments of a method of identifying cytotoxic T cell activity due to cancer immunotherapy in a subject are provided herein. The method includes administering an effective amount of a tracer for PET or SPECT to a subject receiving a cancer immunotherapy. The tracer can be administered, for example, with, just before (for example an hour before), or after administration of the immunotherapeutic. The tracer comprises an antibody or antigen-binding fragment thereof that specifically binds to a luminal domain of a LGAM and is labeled with a PET or SPECT detectable moiety. The signal of the tracer in the subject is detected by PET or SPECT to identify the cytotoxic T cell activity due to immune therapy for the cancer in the subject.

As discussed herein, during T-cell degranulation elicited by cancer immunotherapy, LGAMs including CD107a, CD107b, and CD63 are present on the surface of the T-cell, a substantial portion of which are endocytosed back into intracellular vesicles. Binding of the labeled antibody or antigen binding fragment to the cell surface LGAM results in internalization of the labeled antibody or antigen binding fragment. This concentrates the antibody or antigen binding fragment (and the corresponding PET or SPECT detectable moiety) within cells with surface expression of the LGAM (that is, degranulating T cells), and allows in vivo detection of such cells by PET or SPECT imaging. If the cancer immunotherapy fails to elicit cytotoxic T-cell activity at the site of the tumor, then cell-surface LGAM marker is not present (or is minimally present) and the antibody or antigen binding fragment (and the corresponding PET or SPECT detectable moiety) is not concentrated at the tumor site. Thus, the presence (or absence) of PET/SPECT detection in the location of the cancer can be used to identify cytotoxic T cell activity due to cancer immunotherapy, which information can be used to evaluate effectiveness of the cancer immunotherapy.

Accordingly, in several embodiments, the method is used to identify subjects responding or not responding to a cancer immunotherapy. In some embodiments, the method is used to monitor a subject receiving a cancer immunotherapy to determine whether or not the immunotherapy continues to induce cytotoxic T cell activity at the tumor site in the subject and should be maintained.

In several embodiments, the signal of the PET/SPECT tracer in the subject is quantified and/or localized to identify an amount and/or location of cytotoxic T cell activity in the subject. For example, PET imaging of the subject following administration of the PET tracer can be conducted to detect the presence of the tracer and identify the concentration of PET tracer at relevant location(s) in the subject (such as in a tumor, for example, a glioma).

In some embodiments, detecting an increase in the signal of the PET/SPECT tracer in the subject (for example, at the location of a known tumor in a subject targeted by the cancer immunotherapy) compared to a control identifies the presence of the cytotoxic T cell activity due to the cancer immunotherapy in the subject; and detecting no increase in the signal of the PET/SPECT tracer in the subject (for example at the location of a known tumor in the subject targeted by the cancer immunotherapy) compared to a control identifies a lack of cytotoxic T cell activity due to the cancer immunotherapy in the subject. Any suitable control can be used, for example, a signal of the PET/SPECT tracer based on a location in the subject known to be tumor free, or a standard value, or an amount of the cytotoxic T-cell marker-positive cytotoxic T-cells in a subject that has not been administered the cancer immunotherapy. In some embodiments, the increase in signal can be, for example, at least a 25% increase, at least a 50% increase, at least a 75% increase, at least a 100% increase, or more, compared to a suitable control.

In some embodiments, the method further comprises continuing the cancer immunotherapy if cytotoxic T cell activity due to immune therapy for the cancer is detected in the subject. In some embodiments, the method further comprises stopping or changing the cancer immunotherapy if cytotoxic T cell activity due to immune therapy for the cancer is not detected in the subject.

The method can be utilized with any subject undergoing cancer immunotherapy, including human and veterinary subjects. In some embodiments, the method further comprises selecting the subject receiving the cancer immunotherapy. In some embodiments, the cancer immunotherapy comprises administering to the subject an adoptive cell therapy (such as CAR T-cell therapy, a TCR therapy, or a TIL therapy), NK-cell activating therapy, oncolytic viral therapy, Bi-specific T-cell engager (BiTE) therapy, immunosuppression-targeted immunotherapy (such as CSF-1R inhibitor or IDO inhibitor), immune-sensitizing therapies (such as decitabine or lenalidomide), a tumor vaccine, or an immune checkpoint inhibitor to treat cancer in the subject.

Detection of cytotoxic T cell activity in a subject undergoing cancer immunotherapy can be performed at any suitable time during or after administration of the cancer immunotherapy to the subject. In some embodiments, the method is used to identify cytotoxic T cell activity due to the cancer immunotherapy within a defined time period from initiation of the cancer immunotherapy, such as within 12 weeks (for example, at 12, 10, 8, 6, 4, and/or 2 weeks following initiation of the cancer immunotherapy.

The amount of the labeled antibody or antigen binding fragment that is administered to the subject is effective to specifically bind to the LGAM luminal domain on the surface of cells and be detected by PET or SPECT. Suitable dosages can be readily determined using established approaches to evaluate immunoPET and immunoSPECT tracers, for example, by administering a titrated dosage range in an animal mode to determine an appropriate dosage that maximizes signal and specificity.

The disclosed method can be used to assess response to immunotherapy for any suitable cancer, including but not limited to solid tumors and hematological cancers.

Non-limiting examples of hematological tumors for which the disclosed method may be used include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Non-limiting examples of solid tumors for which the disclosed method may be used sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma including glioblastoma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma). In several examples, the disclosed method is used to identify cytotoxic T cell activity due to cancer immunotherapy for a glioblastoma. In some examples, the disclosed method is used to identify cytotoxic T cell activity due to cancer immunotherapy for colon cancer, glioma, breast cancer, lung cancer, renal cancer, or melanoma. In some examples, the disclosed method is used to identify cytotoxic T cell activity due to cancer immunotherapy for a cancer that has metastasized to a region of the subject that is not accessible by surgery, for example inoperable metasteses of a cancer to the brain of a subject.

In some embodiments, the method is used to detect cytotoxic T cell activity in diseases or conditions other than cancer, such as non-oncologic disease where the cause of disease or therapeutic intervention involves cellular degranulation. In such embodiments, the method is performed on subjects that are not receiving a cancer immunotherapy.

### IV. ImmunoPET and ImmunoSPECT Tracers

Provided are immunoPET and immunoSPECT tracers for use with the method disclosed herein. The tracers include an antibody or antigen-binding fragment thereof that specifically binds to a luminal domain of a LGAM that is labeled with a detectable moiety for PET or SPECT imaging.

Any suitable LGAM-luminal domain-specific antibody or antigen binding fragment can be used. The antibody or antigen binding fragment specifically binds to the luminal domain of the LGAM when present on the cell surface and is internalized along with the LGAM upon cell-surface-endosomal cycling. This concentrates the antibody or antigen binding fragment (and the corresponding PET tracer) within cells with surface expression of the LGAM, and allows in vivo detection of such cells by PET imaging.

In some embodiments, the antibody or antigen binding fragment specifically binds to the luminal domain of CD107a, which is set forth as residues 29-382 of GenBank NP_005552.3, or the luminal domain of CD107b, which is set forth as residues 29-375 of GenBank NP_002285.1:

CAD107a(GenBank NP_005552.3)
(SEQ ID NO: 1)
MAAPGSARRPLLLLLLLLLLGLMHCASAAMFMVKN
GNGTACIMANFSAAFSVNYDTKSGPKNMTFDLPSD
ATVVLNRSSCGKENTSDPSLVIAFGRGHTLTLNFT
RNATRYSVQLMSFVYNLSDTHLFPNASSKEIKTVE
SITDIRADIDKKYRCVSGTQVHMNNVTVTLHDATI
QAYLSNSSFSRGETRCEQDRPSPTTAPPAPPSPSP
SPVPKSPSVDKYNVSGTNGTCLLASMGLQLNLTYE
RKDNTTVTRLLNINPNKTSASGSCGAHLVTLELHS
EGTTVLLFQFGMNASSSRFFLQGIQLNTILPDARD
PAFKAANGSLRALQATVGNSYKCNAEEHVRVTKAF
SVNIFKVWVQAFKVEGGQFGSVEECLLDENSMLIP
IAVGGALAGLVLIVLIAYLVGRKRSHAGYQTI
CD107b (GenBank NP_002285.1)
(SEQ ID NO: 2)
MVCFRLFPVPGSGLVLVCLVLGAVRSYALELNLTD
SENATCLYAKWQMNFTVRYETTNKTYKTVTISDHG
TVTYNGSICGDDQNGPKIAVQFGPGFSWIANFTKA
ASTYSIDSVSFSYNTGDNTTFPDAEDKGILTVDEL
LAIRIPLNDLFRCNSLSTLEKNDVVQHYWDVLVQA
FVQNGTVSTNEFLCDKDKTSTVAPTIHTTVPSPTT
TPTPKEKPEAGTYSVNNGNDTCLLATMGLQLNITQ
DKVASVININPNTTHSTGSCRSHTALLRLNSSTIK
YLDFVFAVKNENRFYLKEVNISMYLVNGSVFSIAN
NNLSYWDAPLGSSYMCNKEQTVSVSGAFQINTFDL
RVQPFNVTQGKYSTAQDCSADDDNFLVPIAVGAAL
AGVLILVLLAYFIGLKHHHAGYEQF

In some embodiments, the antibody or antigen binding fragment is based on the CD107a-specific 1D4B antibody, G1/139/5 antibody, huMAb1 antibody, huMAb2 antibody, or huMAb3 antibody. 1D4B antibody is available, for example, from Developmental Studies Hybridoma Bank (DHSB), University of Iowa and also available from other sources, e.g., Sigma No. MABC39 (see also antibody registry No. AB_2134500). G1/139/5 antibody is available, for example, from Developmental Studies Hybridoma Bank (DHSB), University of Iowa (see also, antibody registry No. AB_10659721). huMAb1, huMAb2, or huMAb3 antibodies are described, for example, in US2018/0142032, which is incorporated by reference herein.

In some embodiments, the antibody used in the disclosed method can be a human antibody or antigen binding fragment thereof. Chimeric antibodies may also be used. The antibody or antigen binding fragment can include any suitable framework region, such as (but not limited to) a human framework region from another source, or an optimized framework region. Alternatively, a heterologous framework region, such as, but not limited to a mouse or monkey framework region, can be included in the heavy or light chain of the antibodies.

The antibody can be of any isotype. The antibody can be, for example, an IgM or an IgG antibody, such as IgG₁, IgG₂, IgG₃, or IgG₄. The class of an antibody that specifically binds to a LGAM (such as CD107a) can be switched with another. In one aspect, a nucleic acid molecule encoding V_{L} or V_{H} is isolated such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved, for example, using a vector or nucleic acid molecule that comprises a C_{L} or C_{H} chain. For example, an antibody that specifically binds the CD107a protein, that was originally IgG may be class switched to an IgM. Class switching can be used to convert one IgG subclass to another, such as from IgG₁ to IgG₂, IgG₃, or IgG₄.

In some examples, the disclosed antibodies are oligomers of antibodies, such as dimers, trimers, tetramers, pentamers, hexamers, septamers, octomers and so on.

Any suitable antigen binding fragment that specifically binds to the luminal domain of a LGAM (such as CD107a) may be used in the disclosed method, such as Fab, F(ab′)₂, and Fv which include a V_{H} and V_{L} and specifically bind a LGAM (such as CD107a), and engineered forms thereof. These antibody fragments retain the ability to selectively bind with the target antigen and are “antigen-binding” fragments. Non-limiting examples of such fragments include:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, a genetically engineered fragment containing the V_{L} and V_{L} expressed as two chains; and

(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.

(6) A multimer of single chain antibodies, for example, expressed as two polypeptide chains [(scFV)₂] or a single polypeptide chain [sc(Fv)2] or linked via a dimer of C_{H}3 domains (minibody), or bispecific forms thereof, such as a Bis-scFv or a diabody.

Any suitable method of producing the above-discussed antigen binding fragments may be used. Non-limiting examples are provided in Harlow and Lane, Antibodies: A Laboratory Manual, 2^{nd}, Cold Spring Harbor Laboratory, New York, 2013.

Antigen binding fragments can be prepared by proteolytic hydrolysis of the antibody or by expression in a host cell of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

In a non-limiting example, a minibody-based probe is produced from a gene encoding the following structure: signal peptide/secretion signal, V_{H} of the LGAM-specific antibody, 18 amino acid (G₄S)₃ linker, V_{L} of the LGAM-specific antibody, IgG hinge domain, IgG CH3 domain, and optionally cleavable purification tag. Diabodies can be generated by replacing the 18-amino acid linker with a 5 amino acid linker (GSSG) including a -GSC sequence of the C-terminus to generate a cys-diabody. Expression of the engineered antibodies may be accomplished in mammalian cells such as in Expi293 cells (ThermoFisher). The antibody fragments are isolated from cell supernatants by affinity chromatography (e.g., Protein L and Ni-NTA) and polished to final purity by SEC. Purity can be determined by SDS-PAGE gel/immunoblot analysis and/or SEC. Affinity measurements (K_{D}) to target LGAM luminal domain are assessed by SPR. Typically, antibodies used for in vivo analysis have a K_{D}<50 nM for target antigen and no more than 10% aggregation or dimerization on SEC (see. e.g., Rudnick S I, et al. Cancer Res. 2011; 71(6):2250-9; Sung C, et al. Cancer Res. 1992; 52(2):377-84).

The LGAM-specific antibody as discussed above can be conjugated to a PET tracer via linker, or the V_{H} and V_{L} of the indicated antibody can be sequenced and cloned into an appropriate production vector (e.g., IgG production vector scFv production vector, minibody production vector, or diabody production vector) for expression in mammalian cells, purification, and linkage to the PET tracer.

To confirm that the PET tracer labeling step does not disrupt antibody binding to target antigen as presented on cells, a modified T-cell degranulation assay can be used (for example, as described by Johnson L D, et al. Journal of Immunology. 2010; 184(10):5604-11), using T-cells isolated from PMEL mice (Jackson Labs). The PMEL strain carries a rearranged T-cell receptor transgene specific for the mouse homolog of human gp100, an enzyme that is expressed gliomas and other tumors. Stimulation of T-cells from these mice with gp100 peptide (1 μg/ml) for 6 hours results in degranulation and surface exposure of CD107a (Johnson L D, et al. Journal of Immunology. 2010; 184(10):5604-11). The PET-labeled antibody or antigen binding fragment is added during added during peptide stimulation, cells washed in PBS, and then assessed for update of the PET tracer. For example, if the PET tracer is a gamma-emitting radiolabel, then the uptake can be assessed using a gamma counter. Cell groups can include cells receiving an acid wash (pH 2.0) to remove extracellular radioligand (to determine surface vs. re-internalized CD107a) and PMEL T-cells stimulated with irrelevant peptide (Ova1257-264), which will not induce CD107a surface expression.

Determination of optimal specific activity and time point for maximum target uptake for the antibody or antigen binding fragment linked to the PET tracer can be assessed using any suitable procedure, for example, blood clearance assays, standard uptake value (SUV) assays at different imaging timepoints (such as 2, 4, 8, and 12 hours post-injection for probes with relatively fast blood clearance, such as minibodies and diabodies or longer for probes with slower blood clearance such as IgG-based probes).

The antibody or antigen binding fragment can be labeled with any PET or SPECT detectable moiety that is suitable for PET or SPECT imaging in a subject (such as a human). In some embodiments, the detectable moiety is a radionuclide that emits a beta plus (positron) particle. Non-limiting examples of radionuclides for PET tracers include ⁸⁹Zr, ⁶⁴Cu, ¹⁸F, ⁶⁸ Ga, ¹¹C, ⁸⁶Y, ¹²⁴I, 2-[¹⁸F]fluoro-2-deoxy-D-glucose (FDG), and 3′-deoxy-3′-[¹⁸F]fluorothymidine (¹⁸FLT). Non-limiting examples of radionuclides for SPECT tracers include ^{99m}Tc, ¹¹¹In, ⁶⁷Ga, ¹⁷⁷Lu, and ¹³¹I.

The procedure for attaching the radionuclide to the antibody or antigen binding fragment varies according to the particular tracer. Antibodies and antigen binding fragments contain a variety of functional groups, such as carboxyl (—COOH), free amine (—NH²), tyrosine (for radioiodinations) or sulfhydryl (—SH) groups, which are available for reaction with a suitable linker for either conjugation of a chelator or direct radiolabeling. Alternatively, the antibody or antigen binding fragment is derivatized to expose or attach additional reactive functional groups. The derivatization may involve attachment of any of a number of known linker molecules, such as those available from Thermo Fisher Scientific, Waltham, Mass. and MilliporeSigma Corporation, St. Louis, Mo. Typically, the linker forms a covalent bond to the antibody or antigen binding fragment, and also binds or covalently bonds with the chelator or directly with the radionuclide. In several embodiments, the linker forms a covalent bond with the antibody or antigen binding fragment, and attaches to a chelator group that complexes a metal radionuclide to label the antibody or antigen binding fragment to form the immunoPET probe. Suitable linkers include, but are not limited to, straight or branched-chain carbon linkers, heterocyclic carbon linkers, or peptide linkers.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody or antigen binding fragment or other polypeptide can be determined.

The average number of detectable moieties per antibody or antigen binding fragment in the tracer can range, for example, from 1 to 20 moieties per antibody or antigen binding fragment. In some embodiments, the average number of detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, detectable moiety per antibody ratio) of a conjugate may be controlled in different ways, for example, by: (i) limiting the molar excess of detectable moiety-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

In addition to conjugation with a chelator or other moiety for radiolabeling, the antibody or antigen binding fragment can be derivatized or linked to another molecule (such as another peptide or protein). In general, the antibody or antigen binding fragment is derivatized such that the binding to the target antigen or radionuclide is not affected adversely by the additional derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

### V. EXAMPLES

The following examples are provided to illustrate particular features of certain embodiments, but the scope of the claims should not be limited to those features exemplified.

### Example 1

### Anti-CD107a ImmunoPET to Detect T Cell Activation In Vivo Following Immunotherapy

This example illustrates the use of anti-CD107a immunoPET to detect T cell activation in a subject following immunotherapy.

Previous work in PET tracer development for monitoring immunotherapy focused on imaging immune cells generally, or T cells as marked by CD8. While these biomarkers may be useful for specific immunotherapies, a reliable and accurate biomarker of lymphocyte degranulation during immunotherapy-induced tumor cell death is more broadly applicable across immunotherapies and cancers. Provided herein is the first demonstration of a lymphocyte degranulation marker to determine early response to immunotherapy by immunoPET. This work shows that a transitory molecular target (CD107a luminal domain) associated with degranulation can be visualized by immunoPET for accurately monitoring immunotherapeutic efficacy, allowing for assessment and alteration of such therapies at a time point earlier than that provided by prior diagnostic modalities.

The orthotopic C57BL/6-syngeneic GL261 model was used in this example. This is a well-established glioma model for immunotherapy (see, e.g., Szatmari et al., Cancer Sci, 97:546-553, 2006; Oh et al., J Transl Med, 12:107, 2014). Briefly, C57BL/6 mice are injected intracranially in a single hemisphere with GL261 cells, which results in animal death in approximately 30 days due to tumor growth.

To generate radiolabeled CD107a antibody, the anti-CD107a antibody Clone 1D4B (Developmental Studies Hybridoma Bank, University of Iowa, also available from multiple commercial sources, see Sigma No. MABC39) was conjugated with NHS-DFO (N-Hydroxysuccinimide—desferrioxamine chelator) in a controlled fashion to achieve approximately 1-3 DFO chelators per antibody, followed by Zr-89 radiolabeling. The level of radiolabeling was assessed by the Zr-89 isotopic dilution method (Holland J P, et al. J Nucl Med. 2010; 51(8):1293-300).

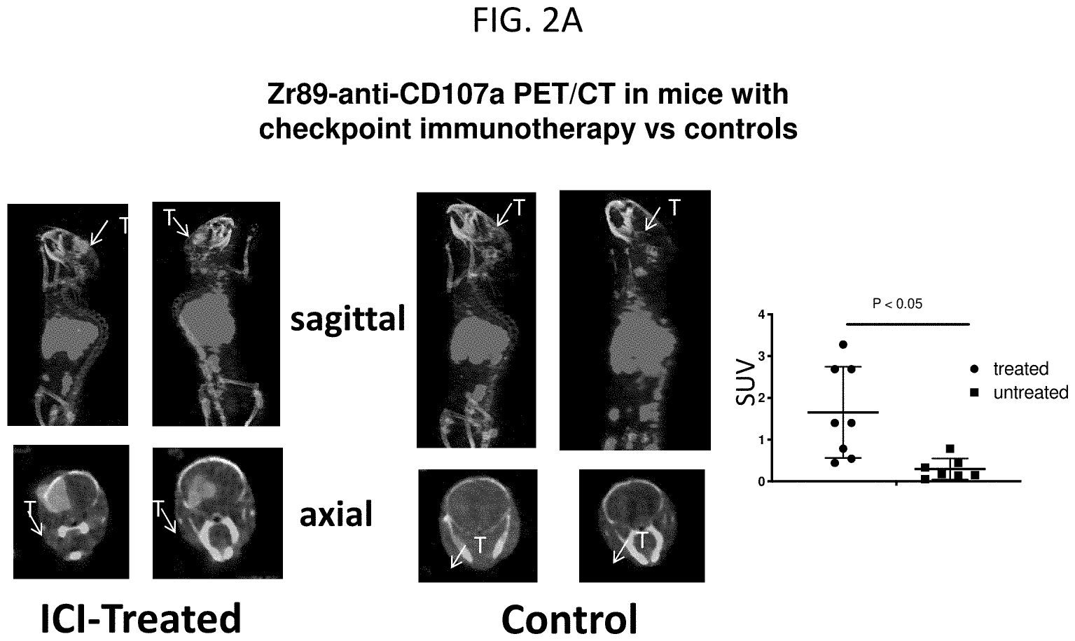

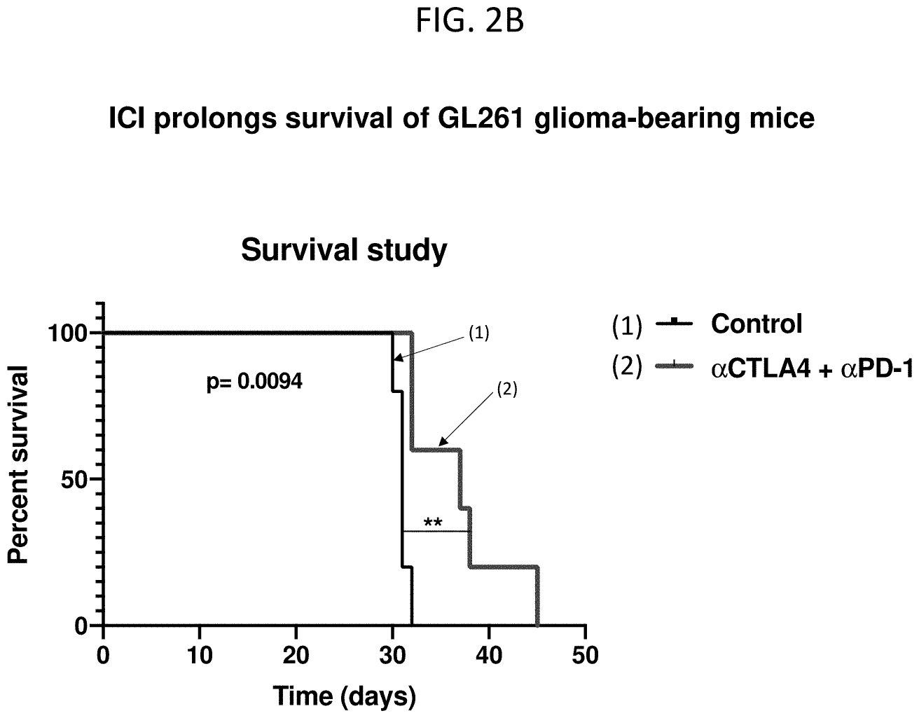

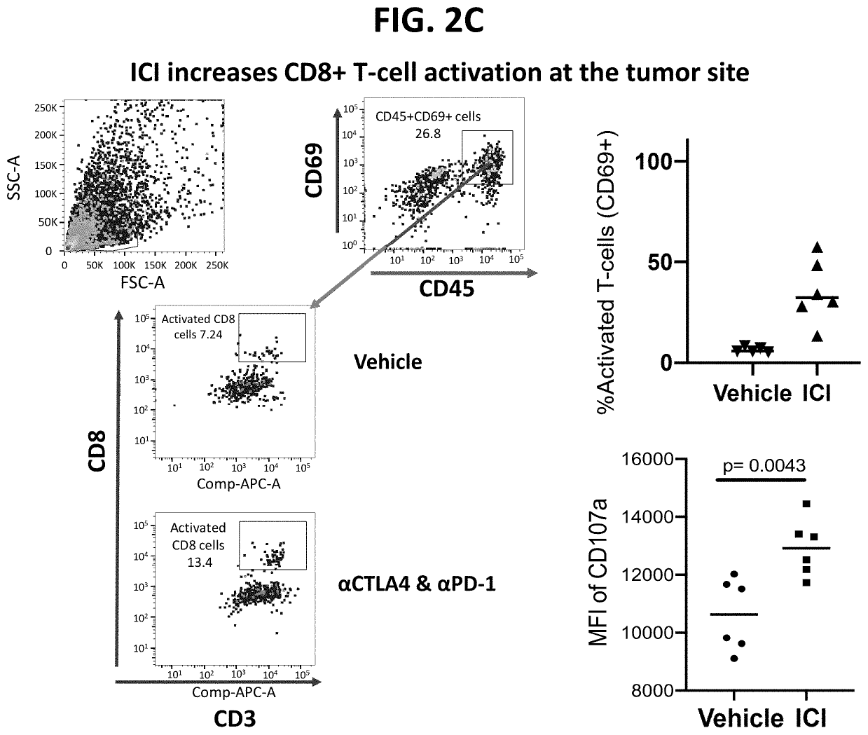

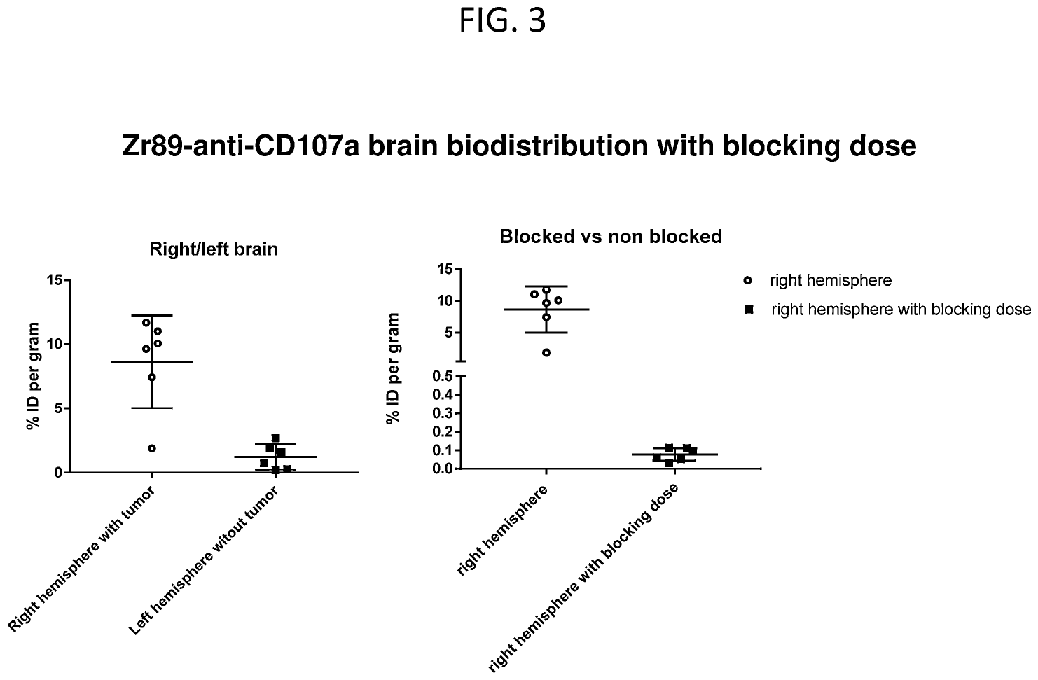

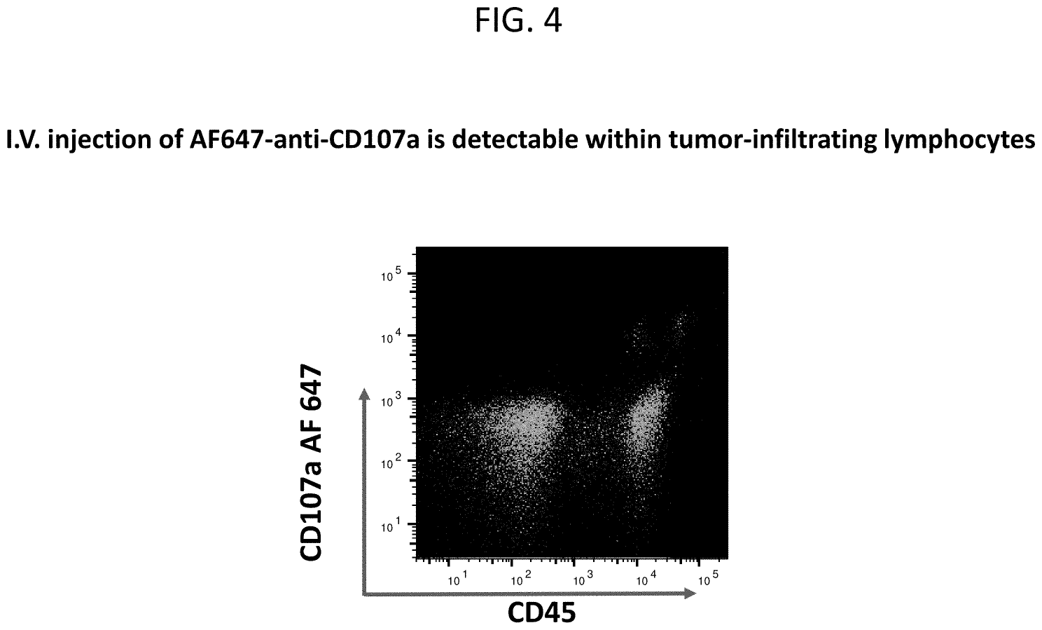

Mice bearing syngeneic GL261 gliomas were treated with either saline (control) or a combination of immune check point inhibitors anti-PD1 and anti-CTLA4 (200 μg of each antibody) immune therapy at 17 and 20 days post tumor-injection (pti). At 20 days pti, the mice were also injected with the ⁸⁹Zr-anti-CD107a probe (100 μCi ⁸⁹Zr-desferrioxamine(DFO)-anti-CD107a, intravenously via tail vein). PET/CT imaging at 23 days pti demonstrated increased tumor uptake of ⁸⁹Zr-CD107a following checkpoint inhibitor immunotherapy (FIG. 2A). Treatment with the immunotherapeutic prolonged mouse survival (FIG. 2B) and increased activated T-cells in tumors (FIG. 2C). Surprisingly, the single time point assessed demonstrated high uptake especially compared to control, with low basal levels of uptake. This was particularly surprising, given the expected low surface receptor density and low cell numbers with surface receptor. A blocking dose (10×) of non-radiolabeled anti-CD107a significantly reduced tracer uptake within tumors (FIG. 3). Additionally, fluorescently labeled (AF-647) anti-CD107a injected into glioma-bearing mice was detected in tumor infiltrating lymphocytes using flow cytometry (FIG. 4).

Taken together, these data demonstrate the effectiveness of the anti-CD107a immunoPET for detecting changes (e.g., an increase) in cytotoxic T cell activity in a subject due to immune therapy. This work shows that CD107a is a surrogate for T-cell degranulation (release of granzyme-B and perforin) during tumor cell killing. Further, these data support that Zr89-DFO-anti-CD107a can be internalized by T cells and detected in vivo shortly after immunotherapy initiation.

### Example 2

### Cu-64 Labeled Anti-CD107a Minibody and Diabody for Monitoring Lytic Degranulation in Gliomas Following Immunotherapy

This example illustrates anti-CD107a minibody (Mb) and diabody (db) antibody fragments as immunoPET agents for identifying cytotoxic T cell activity.

Using the VH and VL sequences of anti-CD107a clone 1D4B (described above), Mb or db genes are generated for incorporation into a vector for transient mammalian cell production. The minibody vector encodes the following structure: mouse Ig Kappa secretion signal, V_{H}, 18 amino acid (G₄S)₃ linker, V_{L}, murine IgG₂ hinge, murine IgG₂ₐ CH3 domain, and His-tag cloned into the mammalian expression pcDNA3.4 vector. The diabody vector structure is the same as the minibody, with replacement of the 18-amino acid linker with a 5 amino acid linker (GSSG) including a -GSC sequence of the C-terminus to generate a cys-db. Expression of the engineered antibodies is performed in Expi293 cells (ThermoFisher). The antibody fragments are isolated from cell supernatants by affinity chromatography (Protein L and Ni-NTA) and polished to final purity by SEC. Purity is determined by both SDS-PAGE gel/immunoblot analysis and SEC, and affinity measurements (K_{D}) to a CD107a ectodomain obtained by SPR.

For radiolabeling of the minibody, NHS-NODA-GA is conjugated in a controlled fashion to the minibody to achieve approximately 1-3 NODA-GA per Mb. For radiolabeling of the diabody, the cysteine double bond is reduced (TCEP) and conjugated with maleimide-NODA-GA. NODA-GA levels are assessed by titration against Cu-arsenazo III (De Silva R A, et al. Nuclear Medicine and Biology. 2012; 39(8):1099-104). The fragments are labeled with Cu-64, as described (Kim H-Y, et al. PloS one. 2018; 13(3):e0192821). Cu-64 (t1/2=12.7 h) enables weekly PET imaging, which is not feasible with Zr-89.

It will be apparent that the precise details of the methods or compositions described may be varied or modified without departing from the spirit of the described embodiments. We claim all such modifications and variations that fall within the scope and spirit of the claims below.

We claim:

1. A method for identifying cytotoxic T cell activity due to cancer immunotherapy in a subject, comprising:
administering an effective amount of a tracer for positron emission tomography (PET) or single photon emission computed tomography (SPECT) to a subject receiving a cancer immunotherapy, wherein the tracer comprises an antibody or antigen-binding fragment thereof that specifically binds to a luminal domain of a lymphocytic granule-associated molecule (LGAM) labeled with a PET or SPECT detectable moiety; and
detecting the signal of the tracer by PET or SPECT to identify the cytotoxic T cell activity due to immune therapy for the cancer in the subject.

2. The method of claim 1, comprising quantifying and localizing the signal of the tracer in the subject to identify the cytotoxic T cell activity due to immune therapy for the cancer in the subject.

3. The method of claim 1, wherein:
detecting an increase in the signal of the tracer in the subject compared to a control identifies the presence of cytotoxic T cell activity due to the cancer immunotherapy in the subject; and
detecting no increase in the signal of the tracer in the subject compared to a control identifies a lack of cytotoxic T cell activity due to the cancer immunotherapy in the subject.

4. The method of claim 3, wherein the control is a standard value.

5. The method of claim 4, wherein the control is an amount of the cytotoxic T-cell marker-positive cytotoxic T-cells in a subject that has not been administered the cancer immunotherapy.

6. The method of claim 1, further comprising continuing the cancer immunotherapy if the presence of cytotoxic T cell activity due to immune therapy for the cancer is detected in the subject.

7. The method of claim 1, further comprising stopping the cancer immunotherapy if cytotoxic T cell activity due to immune therapy for the cancer is not detected in the subject.

8. The method of claim 1, further comprising selecting the subject receiving the cancer immunotherapy.

9. The method of claim 1, wherein the cancer immunotherapy is for treatment of colon cancer, glioma, breast cancer, lung cancer, renal cancer, or melanoma.

10. The method of claim 9, wherein the glioma is glioblastoma.

11. The method of claim 1, wherein the cancer immunotherapy comprises administering to the subject an adoptive cell therapy, a tumor vaccine, or an immune checkpoint inhibitor to treat cancer in the subject.

12. The method of claim 11, wherein the adoptive cell therapy is a chimeric antigen receptor (CAR) T-cell therapy, a T-cell receptor (TCR) therapy, or a tumor-infiltrating lymphocyte (TIL) therapy.

13. The method of claim 1, wherein detection of the cytotoxic T-cell marker occurs within twelve weeks after initiation of the cancer immunotherapy to the subject.

14. The method of claim 1, wherein the LGAM is CD63 or a lysosome-associated membrane protein.

15. The method of claim 14, wherein the lysosome-associated membrane protein is CD107a or CD107b.

16. The method of claim 1, wherein the antigen binding fragment of the antibody is a Fv, a Fab, a F(ab′)₂, scFv fused to the Fc, a scFv, a scFv₂, single domain (sdAb), a diabody or a minibody comprising the heavy and light chain variable regions of the antibody.

17. The method of claim 1, where in the antibody or antigen-binding fragment thereof is any one of an anti-CD107a clone 1D4B, clone G1/139/5, clone huMAb1, huMAb2, or huMAb3 or an antigen binding fragment thereof or a humanized or chimeric form thereof.

18. The method of claim 1, wherein the PET or SPECT detectable moiety is a radionuclide that emits a beta plus particle.

19. The method of claim 1, wherein the PET detectable moiety comprises ⁸⁹Zr, ⁶⁴Cu, ¹⁸F, ⁶⁸Ga, ¹¹C, ⁸⁶Y, or ¹²⁴I, and the PET detectable moiety comprises ^{99m}Tc, ¹¹¹In, ⁶⁷Ga, ¹⁷⁷Lu, or ¹³¹I.

20. The method of claim 1, wherein the subject is a human subject.
